SEQUENCE LISTING (1 sequence)

SEQ ID NO: 1 - protein, 60 aa, Homo sapiens
MAKKSAENGIYSVSGDEKKGPLIAPGPDGAPAKGDGPVGLGTPGGRLAVPPRETWTRQMD

(19) United States
(12) Patent Application Publication
(10) Pub. No.: US 2012/0315654 A1
(43) Pub. Date: Dec. 13, 2012

(54) COMPOSITIONS AND METHODS FOR DETECTING CARDIOTOXICITY

(75) Inventors: Lucia A. Santacruz Kozarinas, Chapel Hill, NC (US); Danny O. Jacobs, Durham, NC (US); James L. Burchette, JR., Durham, NC (US)

(73) Assignee: DUKE UNIVERSITY, Durham, NC (US)

(21) Appl. No.: 13/492,072

(22) Filed: Jun. 8, 2012

Related U.S. Application Data
(60) Provisional application No. 61/494,654, filed on Jun. 8, 2011.

(51) Int. Cl. G01N 33/566; G01N 21/64; G01N 21/76; G01N 33/577

(52) U.S. Cl. 435/7.25; 435/7.21

(57) ABSTRACT

The present disclosure provides methods and compositions for detecting the presence and activity of creatine transporter proteins (CrT) in biological samples comprising screening the samples for CrT using antibodies that bind to the CrT. The methods are useful to detect the onset of cardiotoxicity in subjects undergoing treatment with anthrocyclines or those susceptible to heart-related conditions. The methods and compositions described herein in can be practiced with diagnostic kits.

### CROSS REFERENCE TO RELATED APPLICATION

This application claims the benefit of U.S. provisional application No. 61/494,654, filed Jun. 8, 2011, the disclosure of which is hereby incorporated by reference in its entirety.

### FIELD

The presently disclosed subject matter relates to compositions and methods for detecting cardiotoxicity, and more specifically, for determining a reduction in the presence and/or function of creatine transporter protein.

### BACKGROUND

Anthracyclines are a family of drugs that are effective anti-neoplastic agents, and are commonly used to treat cancer, including leukemia, soft tissue sarcomas, and breast and lung cancer. Anthracyclines intercalate into DNA and are described as topoisomerase Type II poisons. The anthracycline family comprises, daunomycin, daunorubicin, doxorubicin/adriamycin, epirubicin, and idarubicin. While the anthracyclines are known to be potent anti-tumor drugs, their use has been limited due to potentially life-threatening cardiotoxicity associated therewith. This problem may be described as cumulative dose-dependent cardiotoxicity, which can ultimately result in congestive heart failure.

Profound alterations in myocellular creatine and phosphocreatine levels are observed during human heart failure. To maintain its intracellular creatine stores, cardiomyocytes depend upon a cell membrane creatine transporter whose regulation is not clearly understood. Creatine transport capacity in cardiac myocytes is modulated by substrate availability and AMPK (see, e.g., Darrabie, M. D. et al., Am. J. Physiol. Endocrinol. Metab. 300: E870-876, 2011), and it is reduced in the failing myocardium, likely adding to the energy imbalance that characterizes heart failure.

Doxorubicin (DOX) is one of the most effective chemotherapeutic agents used to treat leukemia, lymphomas, and solid tumors such as breast cancer or soft-tissue sarcomas. A major side effect of the drug is dose-dependent cardiotoxicity, which may lead to chronic cardiomyopathy and congestive heart failure. DOX's antitumor activity is thought to be primarily due to DNA damage by intercalation, breakage, alkylation, crosslinking, and inhibition of topoisomerase II (Minotti G. et al., Pharmacol Rev 56: 185-229, 2004; Takemura G. & Fujiwara H., Prog Cardiovasc Dis 49: 330-352, 2007). Although all the mechanisms responsible for DOX's cardiotoxic effects are not yet completely understood, alterations in energy metabolism appear to be important intermediaries (Tokarska-Schlattner M. et al., J Mol Cell Cardiol 41: 389-405, 2006). Crucial components of cardiac energy metabolism, such as AMPK signaling pathways, fatty acid metabolism, mitochondrial oxidation, and creatine kinase function are perturbed (Tokarska-Schlattner M. et al., 2006). Creatine (Cr) and its phosphorylated form phosphocreatine (PCr), together with creatine kinases (CK), comprise a system that helps maintain ATP stores in cardiac and skeletal myocytes (Wyss M. & Kaddurah-Daouk R., Physiol Rev 80: 1107-1213, 2000).

The myocardium's main source of Cr is provided by transmembrane uptake via a specific Cr transporter (CrT), which belongs to the SLC6 family of membrane proteins (Nash S. et al., Receptors Channels 2: 165-174, 1994). One cell line that is useful for studying the effects of DOX on CrT function is rat neonatal cardiomyocytes (RNCM), as this line is a primary cardiac cell culture, and thus possesses native cellular signaling cascades. RNCM preferentially use glucose and lactate as energy sources over fatty acids, the main energy substrate of adult cardiomyocytes (Lopaschuk G. D. & Jaswal J. S., J Cardiovasc Pharmacol 56: 130-140, 2010). In addition, HL-1 cells are a well-established immortalized murine atrial cell line that retain the essential hallmarks of primary cardiomyocytes, and are a proven expression system to study protein structure, function and modulation of cardiomyocytes (White S. M. et al., Am J Physiol Heart Circ Physiol 286: H823-829, 2004). HL-1 cells have very low native Cr transport and, thus, are ideally suited to study Cr transport following transfection with a mammalian expression vector encoding the human CrT.

Accordingly, it is an object of the present disclosure to provide methods for detecting the onset of cardiotoxicity in subjects undergoing treatment with anthrocyclines or those susceptible to heart-related conditions.

### SUMMARY

One aspect of the present disclosure provides methods for detecting cardiotoxicity in a subject undergoing treatment with an anthracycline compound comprising contacting a biological sample obtained from the subject with a monoclonal antibody that is specific for the creatine transporter protein; and determining the level of binding of the antibody to the creatine transporter protein in the biological sample, wherein a lowered binding of antibody as compared to a control is indicative of cardiotoxicity.

Another aspect of the present disclosure provides a method for detecting cardiotoxicity in a subject susceptible to a heart-related condition comprising contacting a biological sample obtained from the subject with a monoclonal antibody that is specific for the creatine transporter protein; and determining the level of binding of the antibody to the creatine transporter protein in the biological sample, wherein a lowered binding of antibody as compared to a control is indicative of cardiotoxicity.

Another aspect of the present disclosure provides a kit for determining the likelihood of the presence of cardiotoxicity in a subject, the kit comprising (i) an antibody that specifically binds to the creatine transporter protein and (ii) instructions for use in a method of determining the likelihood of the presence of cardiotoxicity in a subject, the method comprising contacting a biological sample obtained from the subject with a monoclonal antibody that is specific for the creatine transporter protein and determining the level of binding of the antibody to the creatine transporter protein in the biological sample, wherein a lowered binding of antibody as compared to a control is indicative of cardiotoxicity.

In certain embodiments, the antibody comprises antibody produced by hybridoma cell line 4B9. In other embodiments, the antibody comprises antibody produced by hybridoma cell line 8A6. In other embodiments, the antibody recognizes the isoform of creatine transporter protein that is recognized by the antibody produced by hybridoma cell line 4B9 or 8A6. In other embodiments, the antibody is specific for an epitope in the first 60 amino acids of the human creatine transporter protein (SEQ ID NO: 1).

In other embodiments, the level of antibody binding is determined by flow cytometry, immunohistochemistry, ELISA or Western blotting.

In yet another embodiment, the control is a control sample obtained from a biological sample from an individual not undergoing treatment with an anthracycline compound, the method comprising contacting the control sample with the monoclonal antibody that is specific for the creatine transporter protein; and determining the level of binding of the antibody to the creatine transporter protein in the control sample.

In other embodiments, the biological sample comprises blood or body tissue.

Another aspect of the present disclosure provides a method for detecting the onset of cardiotoxicity in a subject undergoing treatment with an anthracycline compound comprising obtaining a blood sample from a subject prior to treatment of the subject with an anthracycline compound and measuring creatine transporter protein activity in the blood sample erthyrocytes; obtaining a blood sample from the subject after receiving at least one treatment with the anthracycline compound and measuring creatine transporter protein activity in the blood sample erthyrocytes; comparing the level of creatine transporter protein activity in the erthyrocytes, wherein a reduced creatine transporter protein activity in the erthyrocytes after treatment with the anthracycline compound is indicative of the onset of cardiotoxicity; and ceasing treatment with the anthracycline compound when the reduced creatine transporter protein activity is observed.

Other aspects and advantages will become apparent to those skilled in the art from a review of the following description that proceeds with reference to the following illustrative drawings.

### BRIEF DESCRIPTION OF THE DRAWINGS

The accompanying drawings are not intended to be limiting in any way. The foregoing objects, features and advantages of the present disclosure will become more apparent from a reading of the following description in connection with the accompanying drawings.

FIG. 1A and FIG. 1B show that doxorubicin (DOX) decreases Cr transport in rat neonatal cardiac myocytes (RNCM). FIG. 1A shows RNCM cells grown in media containing 100 nmol/L DOX for the indicated times. Control values were determined from transport assays with cells incubated in media devoid of DOX. FIG. 1B shows RNCM cells incubated with 100 nmol/L DOX for 4 hrs, followed by incubation in normal media (devoid of DOX). Data points represent ¹⁴C—Cr transport measured in quadruplicate in 5 independent experiments. An “*” indicates a statistically significant decrease in transport in comparison to cells grown in control media (ANOVA, p<0.05, Fisher's LSD).

FIG. 2A and FIG. 2B show a time course and dose-response effects of DOX on HL-1 cells. HL-1 cells expressing the human CrT protein were incubated in media containing increasing concentrations of DOX during 24 hrs (FIG. 2A) or in 100 nmol/L DOX for 12, 24, 36, and 48 hrs (FIG. 2B). Cr transport was measured as described herein in Example 1. Data correspond to 4 separate experiments, each done in triplicate.

An “*” indicates a statistically significant decrease in transport in comparison with controls (ANOVA p<0.05, Fisher's LSD).

FIG. 3A and FIG. 3B show that significant changes in Cr transport precede DOX induced apoptosis and cytotoxicity in RNCM and HL-1 cells. Caspase 3/7 activation (FIG. 3A) or LDH release (FIG. 3B) were measured in RNCM and HL-1 cells, incubated in 100 nmol/L DOX for increasing periods of time. A statistically significant increase in apoptosis was not observed. LDH release was significantly elevated after incubation with 100 nmol/L DOX for 24 hrs (*) in HL-1 cultures (hollow circles), or after 36 hrs (t) in RNCM cultures (filled circles, p<0.05, ANOVA, Fisher's LSD). Data correspond to 7 separate experiments. LDH release was not significantly elevated in HL-1 or RNCM cells incubated with 50 nmol/L DOX through 48 hrs of observation (FIG. 3B insert, n=3).

FIG. 4 shows immunoblots of CrT protein expressed in HL-1 cells. Representative Western blots of solubilized protein isolated from HL-1 cells expressing a negative control, CrT, CrT-myc or a rat-human CrT chimera are shown. The immunoblot was probed with one of 2 distinct anti-human CrT rat monoclonal antibody clones (4B9 or 8A6), or myc tag antibody, as described herein in Examples 1 and 2. Equal amounts (25 μg) of protein were loaded in each lane. TATA binding protein (TBP) was used as a loading control.

FIG. 5A and FIG. 5B show that DOX reduces cell-surface CrT protein content in HL-1 cells. FIG. 5A shows the abundance of CrT cell-surface protein that was analyzed using cell biotin labeling, followed by isolation using avidin affinity and Western blots probed with 4B9 antibody. FIG. 5B shows the intensity of the bands quantified using IMAGE J. Data represent the mean±SEM of 4 independent experiments, and an “*” indicates a significant difference compared with the controls (p<0.05, ANOVA, Fisher's LSD).

FIG. 6 shows immunohistochemistry slides showing heart tissue from transgenic mice overexpressing the human creatine transporter protein (CrT) stained with hematoxylin/eosin (H/E), negative control (no antibody), or 4B9 antibody at 1:500 and 1:50 dilutions, as described herein in Example 2.

FIG. 7 shows immunohistochemistry slides showing human heart tissue stained with either hematoxylin/eosin (H/E), 4B9, or 8A6 antibody, as described herein in Example 2.

FIG. 8 shows immunohistochemistry slides of normal cardiac muscle and failing cardiac muscle stained with either hematoxylin/eosin (H/E), negative control (no antibody), or 4B9 antibody at a 1:25 dilution, as described herein in Example 2.

FIG. 9 shows creatine transport measured in human erythrocytes.

FIG. 10 shows immunoflorescent labeling of human erythrocytes with 8A6 antibody.

FIG. 11 shows human erythrocyte immunofluorescence using 4B9 antibody.

### DETAILED DESCRIPTION

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Articles “a” and “an” are used herein to refer to one or to more than one (i.e. at least one) of the grammatical object of the article. By way of example, “an element” means at least one element and can include more than one element.

Unless otherwise defined, all technical terms used herein have the same meaning as commonly understood by one of ordinary skill in the art to which this disclosure belongs.

In general, the methods described herein include evaluating the levels of human creatine transporter protein (CrT) in a biological sample from a subject. These levels can provide diagnostic and/or prognostic information, e.g., indicating whether a subject has onset of cardiotoxicity, either caused by anthracycline treatment or other heart-related conditions.

In one embodiment of the presently disclosed subject matter, a method is provided for detecting cardiotoxicity in a subject undergoing treatment with an anthracycline compound comprising: contacting a biological sample obtained from the subject with a monoclonal antibody that is specific for the creatine transporter protein; and determining the level of binding of the antibody to the creatine transporter protein in the biological sample; wherein a lowered binding of antibody as compared to a control is indicative of cardiotoxicity.

In one embodiment, the control is a control sample obtained from a biological sample from an individual not undergoing treatment with an anthracycline compound, the method comprising contacting the control sample with the monoclonal antibody that is specific for the creatine transporter protein; and determining the level of binding of the antibody to the creatine transporter protein in the control sample. In one embodiment, the control is the biological sample obtained from the individual prior to beginning treatment with the anthracycline compound.

In one embodiment of the presently disclosed subject matter, a method is provided for detecting cardiotoxicity in a subject susceptible to a heart-related condition comprising: contacting a biological sample obtained from the subject with a monoclonal antibody that is specific for the creatine transporter protein, and determining the level of binding of the antibody to the creatine transporter protein in the biological sample, wherein a lowered binding of antibody as compared to a control is indicative of cardiotoxicity.

In one embodiment the control is a sample obtained from a biological sample from an individual not susceptible to a heart-related condition, the method comprising contacting the control sample with the monoclonal antibody that is specific for the creatine transporter protein, and determining the level of binding of the antibody to the creatine transporter protein in the control sample.

Hence, changes in creatine metabolism manifested through alterations in creatine transport capacity, i.e., changes in creatine transporter protein abundance and/or function, can be a useful diagnostic or predictive tool in monitoring the onset of cardiotoxicity in a subject.

As used herein, the term “anthracycline compound” refers to the class of drugs used in cancer chemotherapy derived from Streptomyces bacteria. Examples include, but are not limited to, daunorubicin, doxorubicin (Adriamycin), Epirubicin, Idarubicin, Valrubicin and Mitoxantrone. The term “anthracycline treatment” refers to the use of these compounds in a cancer regimen.

As used herein, the term “cardiotoxicity” refers to any condition where there is damage to the heart muscle. Such damage may be caused by chemotherapy drugs (e.g., anthracycline compounds) or other medications, as well as cardiac muscle damage caused by other conditions, such as hypertrophy, ischemia, ischemia-reperfusion, hypoxia and the like commonly associated with cardiac arrest, heart failure, etc.

As used herein, the term “heart-related conditions” refers to those conditions associated with, or prognosticator of, damage to the heart muscle. Such conditions include, but are not limited to, high blood pressure, high cholesterol, smoking, diabetes, age, race and the like.

As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like. Preferably, the subject is a human patient. More preferably, the subject is a human patient undergoing treatment with an anthracycline compound or is susceptible to a heart-related condition.

Thus, for a subject who has low levels of CrT as compared to controls, there is a higher probability of cardiotoxicity. Conversely, for a subject who has normal levels of CrT as compared to controls, there is a low probability of cardiotoxicity. For example, a subject undergoing a cancer treatment regimen with an anthracycline compound that exhibits a low CrT level suggests that anthracycline-induced cardiotoxicity is highly probable. This diagnosis therefore allows a caregiver to alter and/or stop treatment before a cardiotoxic event takes place. Such action allows for the caregiver to design and implement a “personalized” anthracyclin compound treatment regimen. Similarly, a subject that is susceptible to a heart-related condition that exhibits a low CrT level would indicate that a cardiotoxic event is imminent, thereby allowing the caregiver to take appropriate action.

Evaluating the levels of CrT in a subject typically includes obtaining a biological sample. As used herein, the term “biological sample” refers to any sample that can be obtained from the subject that comprises CrT protein. Such samples include, but are not limited to, serum, blood, plasma, urine, or body tissue (e.g., a biopsied tissue specimen). Levels of the CrT protein in the sample can be determined by measuring levels of the CrT protein in the sample using methods known in the art and/or described herein, e.g., immunoassays such as enzyme-linked immunosorbent assays (ELISA), immunohistochemistry, Western blotting and the like. In one embodiment, the sample is a blood sample, and the levels of CrT protein are determined for the erythrocytes from the blood sample.

For example, a method described herein, e.g., for levels of CrT in a subject undergoing treatment with an anthracycline compound, can include contacting a sample from the subject, e.g., a sample including blood, serum, plasma, urine, or body tissue, from the subject with an antibody that specifically binds to the CrT protein as described herein. The methods can also include contacting a sample from a control subject, normal subject, or normal tissue or fluid from a normal subject with the antibody to provide a reference or control. Moreover, the method can additionally include comparing the specific binding of the antibody to the test subject with the specific binding of the antibody to the normal subject, control subject, or normal tissue or fluid from the normal or control subject. Expression from a control subject or control sample can be provided as a predetermined value, e.g., acquired from a statistically appropriate group of control subjects.

An antibody that “binds specifically to” the CrT protein, binds preferentially to the CrT protein in a sample containing other proteins. The term “antibody” as used herein refers to an immunoglobulin molecule or immunologically active portion thereof, i.e., an antigen-binding portion. Examples of immunologically active portions of immunoglobulin molecules include F(ab) and F(ab′)₂ fragments which can be generated by treating the antibody with an enzyme, such as pepsin. The antibody can be polyclonal, monoclonal, recombinant, e.g., a chimeric or humanized, fully human, non-human, e.g., murine, monospecific, or single chain antibody. In one embodiment, it has effector function and can fix complement. In one embodiment, the antibody is a rat monoclonal antibody specific for the CrT protein. In one embodiment, the antibody is 4B9 or 8A6. In one embodiment, the antibody recognizes the isoform of creatine transporter protein that is recognized by the antibody produced by hybridoma cell line 4B9 or 8A6. In one embodiment, the antibody is generated by using the first 60 amino acids of the human creatine transporter protein (SEQ ID NO: 1) as the antigen. In one embodiment, the antibody is specific for an epitope in the first 60 amino acids of the human creatine transporter protein (SEQ ID NO: 1). In one embodiment, the antibody comprises antibody produced by hybridoma cell line 4B9 or hybridoma cell line 8A6.

Detection can be facilitated by coupling (i.e., physically linking) the antibody or probe to a detectable substance (i.e., antibody labeling). Examples of detectable substances include various enzymes, prosthetic groups, fluorescent materials, luminescent materials, bioluminescent materials, and radioactive materials. Examples of suitable enzymes include, but are not limited to, horseradish peroxidase, alkaline phosphatase, β-galactosidase, or acetylcholinesterase; examples of suitable prosthetic groups include, but are not limited to, complexes such as streptavidin/biotin and avidin/biotin; examples of suitable fluorescent materials include, but are not limited to, umbelliferone, fluorescein, fluorescein, isothiocyanate, rhodamine, dichlorotriazinylamine fluorescein, dansyl chloride, quantum dots, or phycoerythrin; an example of a luminescent material includes, but is not limited to, luminol; examples of bioluminescent materials include, but are not limited to, luciferase, luciferin and acquorin, and examples of suitable radioactive materials include, but are not limited to, ¹²⁵I, ¹³¹I, ³⁵S or ³H.

Diagnostic assays can be used with biological matrices such as live cells, cell extracts, cell lysates, fixed cells, cell cultures, bodily fluids, of forensic samples. Conjugated antibodies useful for diagnostic or kit purposes, include antibodies coupled to dyes, isotopes, enzymes, and metals, see, e.g., Le Doussal et al., New Engl. J. Med. 146:1:69-175 (1991); Gibellini et al., J. Immunol. 160:3891-3898 (1998); Hsing & Bishop, New Engl. J. Med. 162:2804-2811 (1999); Everts et al., New Engl. J. Med. 168:883-889 (2002), the entire contents of which are hereby incorporated by reference. Various assay formats exist, such as radioimmunoassays (RIA), ELISA, and lab on a chip (U.S. Pat. Nos. 6,176,962 and 6,517,234, the entire contents are hereby incorporated by reference).

In one embodiment, the presently disclosed subject matter provides a kit for determining the likelihood of the presence of cardiotoxicity in a subject, the kit comprising (i) an antibody that specifically binds to the creatine transporter protein and (ii) instructions for use in a method of determining the likelihood of the presence of cardiotoxicity in a subject, the method comprising: contacting a biological sample obtained from the subject with a monoclonal antibody that is specific for the creatine transporter protein; and determining the level of binding of the antibody to the creatine transporter protein in the biological sample, wherein a lowered binding of antibody as compared to a control is indicative of cardiotoxicity.

The diagnostic kits of the present disclosure can be configured for professional (e.g., clinical) or personal use. In one embodiment, the kit includes an antibody for the detection of CrT protein, as well as a reagent comprising a binding composition for the detection of the CrT protein, and instructions for use in a method described herein. A control can also be included, e.g., an epitope of CrT. In one embodiment, the kit comprises one or more of a buffer, a secondary antibody, or a detection reagent.

Kits generally comprise the following major elements: packaging, reagents comprising antibodies described above, optionally a control, and instructions. Packaging may be a box-like structure for holding a vial (or a number of vials) containing the antibodies, a vial (or number of vials) containing a control, and instructions for use in a method described herein. Individuals skilled in the art can readily modify the packaging to suit individual needs. The foregoing kits can also be provided with tools to assist in the collection of biological samples. Some samples of tools include sample containers, such as vials or cups; needles and syringes for blood or serum samples; antiseptic prepatory pads; gauze pads; and/or swabs for tissue samples.

As one example, the kit can contain an antibody that binds specifically to CrT (e.g., 4B9 and/or 8A6), or an antibody or antigen binding fragment thereof that binds specifically to the CrT protein. In one embodiment, the antibody recognizes the isoform of creatine transporter protein that is recognized by the antibody produced by hybridoma cell line 4B9 or 8A6. In one embodiment, the antibody is generated by using the first 60 amino acids of the human creatine transporter protein (SEQ ID NO: 1) as the antigen. In one embodiment, the antibody is specific for an epitope in the first 60 amino acids of the human creatine transporter protein (SEQ ID NO: 1). In one embodiment, the antibody comprises antibody produced by hybridoma cell line 4B9 or hybridoma cell line 8A6, or an antibody or antigen binding fragment thereof that binds specifically to the CrT protein.

In other embodiments, other methods of detection can be used, e.g., colorimetric assays, radioimmunoassays, or chemiluminescent assays. Sandwich assays can be used as well, e.g., using two monoclonal antibodies, one labeled with ¹²⁵I and the other adsorbed onto beads (see, e.g., the IRMA-BNP2 kit from CISBIO International (France) and SHIONORIA BNP or ANP kits (SHIONOGI USA, Inc.).

In one embodiment, the presently disclosed subject matter provides a method for detecting the onset of cardiotoxicity in a subject undergoing treatment with an anthracycline compound comprising: obtaining a blood sample from a subject prior to treatment of the subject with an anthracycline compound and measuring creatine transporter protein activity in the blood sample erthyrocytes; obtaining a blood sample from the subject after receiving at least one treatment with the anthracycline compound and measuring creatine transporter protein activity in the blood sample erthyrocytes; comparing the level of creatine transporter protein activity in the erthyrocytes, wherein a reduced creatine transporter protein activity in the erthyrocytes after treatment with the anthracycline compound is indicative of the onset of cardiotoxicity; and ceasing treatment with the anthracycline compound when the reduced creatine transporter protein activity is observed.

The disclosure may be better understood by reference to the following non-limiting Examples, which are provided as exemplary of the disclosure. The following examples are presented in order to more fully illustrate the preferred embodiments of the disclosure and should in no way be construed, however, as limiting the broad scope of the disclosure.

### EXAMPLES

### Example 1

### Effect of Subclinical Concentrations of Doxorubicin on Cardiomyocytes Reduces Creatine Transport

Cell Culture:

Rat neonatal cardiomyocytes (RNCM) were isolated from 1-2 day old Sprague Dawley rats (CHARLES RIVER LABORATORIES, Wilmington, Mass.), and cultured as previously described (Bursac N., et al., Am J Physiol 277: H433-444, 1999). Cells were plated on tissue culture dishes pre-coated with gelatin/fibronectin and allowed to grow in M199 media (INVITROGEN, Carlsbad, Calif.) containing 2% serum before experimentation. All animals were treated according to protocols approved by Duke University's Institutional Animal Care and Use Committee. HL-1 cells (passages 50 to 75), were plated onto fibronectin/gelatin-coated plates and cultured in CLAYCOMB media (SIGMA, St. Louis, Mo.) supplemented with 10% fetal bovine serum, 100 units/ml penicillin, 100 μg/ml streptomycin, 0.1 mM norepinephrine, and 2 mmol/L L-glutamine, as described previously (White S. M., et al., Am J Physiol Heart Circ Physiol 286: H823-829, 2004). The cultures were maintained in a humidified atmosphere of 5% CO₂ at 37° C.

Plasmid Constructs:

Human CrT-cDNA was obtained from Dr. Marc Caron (Duke University, Department of Cell Biology). An EcoR I site followed by a Kozak sequence was added to the 5′ end, and an Hind III site was introduced at the 3′ end and subcloned into the pcDNA3.1(−) mammalian expression vector (INVITROGEN, Carlsbad, Calif.). In a separate construct, an XbaI site was introduced at the 3′ end, removing the natural stop codon. This modified cDNA was then subcloned in the mammalian expression pcDNA 3.1B vector (INVITROGEN, Carlsbad, Calif.). Transfection of this plasmid resulted in expression of CrT protein bearing a C-terminal Myc/His tag that did not alter CrT function and was used to detect the CrT-myc protein by Western blotting. As a negative control for expression, the cDNA encoding CrT was subcloned into pcDNA3.1(+) in reverse orientation with respect to the cytomegalovirus (CMV) promoter.

The rat/human chimeric construct was prepared in two steps using polymerase chain reactions. The first step consisted of two reactions. In the first reaction, the rat N-terminal sequence was amplified using primers that resulted in a 200-bp fragment, including an EcoR I site, a Kozak sequence, and the 5′ end of the cDNA for rat CrT (forward primer: 5′-gaattccaccatggccaagaagagcgcc-3′), with the 3′ end corresponding to the human sequence at position bp167 of the human cDNA (reverse primer: 5′-catgatgaagtccatctgcctggtcca-3′) end. The template for these reactions was a synthetic DNA fragment, encoding the rat CrT N-terminus. A second reaction, using the human CrT-cDNA as template, generated a 700-bp DNA fragment that was complementary at the 5′ end (forward primer: 5′-tggacgcgccagatggacttcatcatg-3′) to the 3′ end of the 200-bp fragment described above, and encompassed a unique Pst I site at position 1104 of the human cDNA (reverse primer: 5′-ccctgactctgccaccttgga-3′).

The products of these two reactions were used as templates in the second step; the forward primer corresponded to the 5′ end of the rat CrT N-terminus, and the reverse primer was the same used in the second reaction of the first amplification round. The resulting 1120-bp fragment was subcloned into the pSCB vector (INVITROGEN, Carlsbad Calif.) and sequenced to verify the integrity of the construct. The EcoR Ito Pst I fragment was isolated and swapped with the corresponding fragment from the human full-length open reading frame. The integrity of the construct was again verified by overlapping sequencing.

CrT Protein Expression in HL-1 Cells:

HL-1 cells have very low native Cr transport, and thus, are well suited for studying Cr transport following transfection with a mammalian expression vector encoding the human CrT (Darrabie M. D., et al., Am J Physiol Endocrinol Metab 300: E870-876, 2011). These cells are a well-established immortalized murine atrial cell line that retain the essential hallmarks of primary cardiomyocytes and are a proven expression system to study protein structure, function, and modulation (Nash S., et al., Receptors Channels 2: 165-174, 1994; White S. M., et al., Am J Physiol Heart Circ Physiol 286: H823-829, 2004).

Transient transfection of HL-1 cardiomyocytes was performed using LIPOFECTAMINE 2000 reagent (INVITROGEN, Carlsbad Calif.) according to the manufacturer's instructions. Cultures intended for Cr transport measurements were grown in 24-well culture plates, whereas those used to characterize the CrT protein were cultured in 6-well dishes. Cells were allowed to grow for approximately 16 hrs after transfection before they were incubated with DOX.

DOX Incubation and ¹⁴C—Cr Transport in RNCM and HL-1 Cells:

100 nmol/L of DOX represents the average steady-state concentration of the drug that is typically measured in patient plasma. For dose-response experiments, cultures were incubated in media containing 25 nmol/L, 50 nmol/L, 75 nmol/L, and 100 nmol/L DOX (SIGMA, St. Louis, Mo.) for 48 hrs. Time course experiments were conducted with cells incubated for 4 hrs, 12 hrs, 24 hrs, 36 hrs, and 48 hrs in media containing 100 nmol/L DOX. To determine the recovery of Cr transport following DOX exposure, cells were first incubated in the presence of 100 nmol/L DOX for 4 hrs. The culture media containing DOX was then removed by aspiration, and the cells were washed with control media. Cr transport assays were performed after growth in control media for 0, 4, 12, 24 or 48 hrs.

Cells used for Cr transport assays were cultured in 24-well plates coated with fibronectin and gelatin. The wells were washed twice with room temperature choline buffer (150 mmol/L choline chloride; 1 mmol/L CaCl₂; 5 mmol/L MgCl₂; 2 mmol/L KCl; and 5 mmol/L HEPES-Tris, pH 7.5). Cells were then incubated for 10 min in a CO₂ incubator at 37° C. in sodium uptake buffer (150 mmol/L NaCl; 1 mmol/L CaCl₂; 5 mmol/L MgCl₂; 2 mmol/L KCl; and 5 mmol/L HEPES-Tris, pH 7.5) that was supplemented with 0.275 μCi/ml of ¹⁴C—Cr (55 mCi/mmol, AMERICAN RADIOLABELED CHEMICALS, St Louis, Mo.) and unlabeled Cr to a final concentration of 15 μmol/L for the dose-response and time course experiments, which is within the reported physiological range (Persky A. M., et al., J Clin Pharmacol 43: 29-37, 2003).

For assays that measured Cr transport kinetics, the final Cr content in the uptake buffer ranged from 5 to 305 μmol/L. Uptake was terminated by aspiration of the radiolabeled solution, followed by 3 washes with ice-cold choline buffer. The cells were lysed with 500 mmol/L NaOH and heated to 80° C. for 30 min. 100 μl of cell lysate was subjected to scintillation counting using a BECKMAN COULTER LS 6500 scintillation counter. Cr transport was normalized to the protein concentration measured using the bicinchoninic acid (BCA) protein assay (PIERCE BIOTECHNOLOGY, Rockford, Ill.).

For experiments with RNCM, each condition was tested in quadruplicate. Measurements were performed in triplicate for experiments performed using HL-1 cells. The number of independent experiments used for data analysis is noted in each corresponding figure legend.

DOX Competition Assays:

For experiments designed to determine if DOX competes with Cr during the transport cycle, uptake assays were performed in the presence of 2 μmol/L DOX, or 2 μmol/L DOX and 100 μmol/L B-GPA, a structural analog of Cr and a inhibitor of Cr transport with an IC₅₀ of 50 μmol/L (Dai W. et al., Arch Biochem Biophys 361: 75-84, 1999). B-GPA and/or DOX were added to the radiolabeled sodium uptake buffer, with Cr concentrations ranging from 5-305 μmol/L. Cells were further processed as described herein above.

Creatine Transporter Protein (CrT) Antibody Generation:

Monoclonal rat anti-human creatine transporter protein (CrT) antibodies were developed using a genetic immunization approach (GENOVAC, Freiburg, Germany). The antigen for the preparation of the antibodies consisted of the first 60 amino acids of the human CrT. A number of positive hybridoma clones were isolated. Two distinct clones (4B9 and 8A6), which only detected the human isoform of CrT, were used for the studies described herein.

Immunoblotting:

HL-1 cells expressing CrT protein were lysed in an ice-cold solution of 150 mmol/L NaCl; 1% Triton X-100; and 50 mmol/L Tris-HCl, pH 7.4, supplemented with protease (COMPLETE MINI protease inhibitors, ROCHE, Indianapolis, Ind.) and phosphatase inhibitors (5 mmol/L NaF; 1 mmol/L phenylmethylsulphonylfluoride; 2.5 mmol/L Na₂P₂O₇; 50 mmol/L β-glycerol; and 1 mmol/L Na₃VO₃, Sigma). Insoluble material was separated by centrifugation at 100,000×g for 30 min at 4° C. Solubilized protein (25 μg per lane) was mixed with Laemmli buffer containing 10% β-mercaptoethanol, separated by SDS-PAGE on a 7% gel for studies of the CrT, and then transferred to nitrocellulose membranes. Blots were incubated with 150 mmol/L NaCl; 0.05% Tween 20; 10 mmol/L Tris-HCl, pH 7.4; and 5% (w/v) low-fat milk powder for at least 1 hrs at room temperature. To detect the CrT protein, the blots were probed with a monoclonal rat anti-human CrT antibody developed using a genetic immunization as described herein above. For CrT-myc protein detection, the blots were probed with anti-myc antibody at a 1:5000 dilution (catalogue #2276, CELL SIGNALING TECHNOLOGY, Danvers, Mass.). The secondary antibody was HRP-conjugated goat anti-rabbit at a 1:10000 dilution. The TATA box-binding protein band was used as a loading control and was detected using a mouse monoclonal antibody (ABCAM, Cambridge, Mass.). An HRP-conjugated goat anti-rat antibody diluted 1:7500 (catalogue #NA 935V GE HEALTHCARE, Piscataway, N.J.) was used as a secondary antibody to detect CrT protein bands. The secondary antibody for detection of the TATA box-binding protein or CrT-myc proteins was an HRP-conjugated anti-mouse antibody (catalogue #NA 931V GE HEALTHCARE, Piscataway, N.J.) diluted 1:10000. Bands were visualized by enhanced chemiluminescence (GE HEALTHCARE LIFE SCIENCES, Piscataway, N.J.). Protein abundance was quantitated by densitometry using the IMAGEJ application from NIH, and normalized to the signal obtained from CrT protein isolated from cells grown in normal culture media.

Cell Surface Labeling:

HL-1 cultures were grown in 75 cm² plates and transiently transfected as described for human CrT cDNA. Cell surface proteins were biotinylated as described by Daniels and Amara (Daniels G. M. & Amara S. G., Methods Enzymol 296: 307-318, 1998) using the cell surface protein isolation kit from PIERCE (Rockford, Ill.). Briefly, cell cultures were washed twice and then resuspended in ice-cold PBS. Cells were lysed with RIPA buffer (150 mmol/L NaCl; 1% deoxycholate; 1% NP-40; 0.1% SDS; and 10 mmol/L Tris-HCl, pH 7.4) containing protease inhibitors for 25 min. Samples were centrifuged at 4° C. for 15 min at 15,000 g. Biotinylated proteins were isolated by incubation of the supernatant with EZ-LINK IMMOBILIZED NEUTRAVIDIN beads (THERMO SCIENTIFIC, Rockford, Ill.) for 60 min at room temperature. After extensive washing, the neutravidin beads were resuspended in Laemmli sample buffer containing 100 mmol/L DTT and eluted at room temperature for one hour. Intracellular (non-biotinylated) CrT protein was isolated from the flowthrough of the NEUTRAVIDIN beads by immunoprecipitation using a specific anti-CrT antibody. Samples were subjected to SDS-PAGE followed by Western blot analysis, as described above. Cell surface CrT protein fraction (biotinylated CrT) was then analyzed by Western blotting using an anti-CrT antibody as described above.

Quantification of Cr, and High-Energy Phosphates:

Tissue culture extracts were prepared using a modification of the method published by Wiseman et al. (Wiseman R. W. et al., Anal Biochem 204: 383-389, 1992). Cultures were grown in 75 cm² cm dishes. After incubation in control or experimental media, the cell monolayer was washed twice and scrapped in ice cold PBS, and were centrifuged 5 minutes at 500×g. The cell pellets were flash frozen in liquid nitrogen and stored at −80° C. until extract preparation. 500 μl of 2N perchloric acid 5 mM EDTA solution was added directly to the tube containing the cell pellets. The solution froze on contact with the pellet and was allowed to thaw to ice/water temperature. The pellet was homogenized, and the slurry was vortexed vigorously followed by centrifugation at 4° C., 20000×g, for 5 minutes. The supernatant was neutralized with an equal volume of 2N KOH, 150 mM TES, and 3M KCl. The potassium perchlorate precipitate was removed by centrifugation at 4° C., 20000×g, 2 minutes. The supernatant was collected and transferred to new tubes and stored at −80° C. AMP, ADP, ATP, and PCr were measured using a modified approach described by Goutier et al (Goutier W. et al, J Neurosci Methods 188: 24-31, 2010). In brief, samples were diluted with mobile phase A (10 mmol/L ammonium bicarbonate buffer adjusted to pH 9.4 with ammonium hydroxide in 20% acetonitrile in HPLC grade water), filtered through a 3000 M.W. cut-off device, and directly injected into a LC-ESI-MS/MS system. Chromatographic separation was accomplished on a SHIMADZU 20A series HPLC (LC) equipped with ZIC-pHILIC (5 μm, 150×4.6) column (SEQUANT, AB, Sweden). Mobile phase B was acetonitrile. Flow rate was 0.8 mL/min. Electrospray ionization (ESI) tandem-mass (MS/MS) detection was performed on an APPLIED BIOSYSTEMS/SCIEX API 4000 QTRAP instrument. The following m/z MS/MS transitions were followed: 346/150.8 (AMP), 426/158.8 (ADP), 506/158.8 (ATP) and 511/158.8 (ATP-¹⁵N₅ (TRC Toronto, Canada), was used as internal standard for ADP, ATP, and PCr). Calibration samples containing each analyte in the range of 0.2-50 μM were run before and after each batch of study samples. Linear relationship signal (analyte/internal standard) vs nominal concentration was found for all the analytes and used for quantification. Cr was measured by a modified creatinine assay (Il'yasova D et al., Cancer Epidemiol Biomarkers Prev 19: 1506-1510, 2010) on the same equipment as for phosphorylated metabolites described above. Briefly, samples were diluted with mobile phase A (10 mmol/L ammonium formate in 0.1% formic acid pH 2.6), filtered through a 3000 M.W. cut-off device, and 10 μL directly injected into the LC-ESI-MS/MS system. Chromatographic separation was accomplished on AGILENT, ZORBAX ECLIPSE PLUS C18, 50×4.6 mm, 1.8 μm column with the following eluents. Mobile phase B: acetonitrile; flow rate: 1 mL/min. The following m/z MS/MS transitions were followed: 114/44 (Cr) and 117/47 (Cr—²H₃. CAMBRIDGE ISOTOPE LABORATORIES, Maine, USA). Calibration samples containing Cr in the range of 0.2-500 won were run before and after each batch of study samples. Linear relationship signal (analyte/internalstandard) vs nominal concentration was found and used for quantification. Under the acidic conditions of the assay PCr undergoes complete hydrolysis to Cr. Thus, measured Cr values were corrected by subtracting the measure PCr levels under basic conditions.

Cytotoxicity and Apoptosis Assays:

RNCM or HL-1 cells transfected with CrT were incubated with 50 or 100 nmol/L DOX for up to 48 hrs. Toxicity was assessed by quantifying the amount of lactate dehydrogenase (LDH) present in the culture media using PROMEGA's CYTOTOX96 NON-RADIOACTIVE ASSAY (PROMEGA, Madison, Wis.). Maximum cytotoxicity was determined by measuring the amount of LDH released in cells incubated with the kit's lysis buffer. The percentage of LDH released after DOX treatment was compared with that of controls.

For apoptosis detection, HL-1 and RNCM cultures were incubated with 100 nmol/L DOX for up to 48 hrs as described above. Apoptosis was measured using PROMEGA's CASPASE-GLO 3/7 ASSAY SYSTEM (PROMEGA). Briefly, attached cells in a single 24-well plate were washed 3 times with ice-cold PBS and resuspended in 500 μl of 1% TRITON-X100; 10% glycerol; 2 mmol/L EDTA; 137 mmol/L NaCl; and 20 mmol/L Tris, pH 8.0. A mixture of 25 μL of lysate and 25 μL of caspase 3/7-assay reagent was prepared, and sequential luminescent readings were taken every 15 min for 4 hrs. Peak signal readings were normalized to protein content and plotted.

Statistical Analyses:

Data are reported as the mean±standard error of the mean. Michaelis-Menten plots were generated using curve-fitting software (SIGMAPLOT, ver. 9.0 San Jose, Calif.). Data were analyzed using nonlinear, least-squared fitting and ANOVA followed by a Fisher's test for pair wise, intergroup comparisons (STASTISTICA, ver. 6.0, Tulsa, Okla.). Correlation was assessed using the Pearson test. Probability values less than 0.05 were considered significant.

DOX Reduced Cr Transport in RNCM:

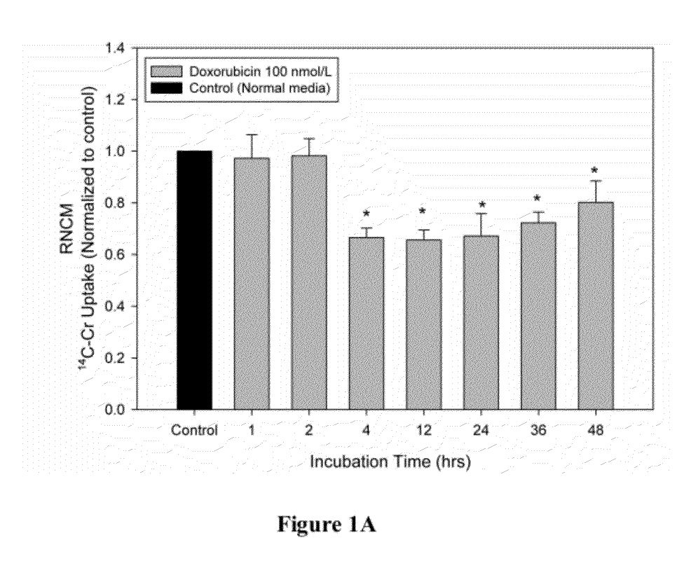

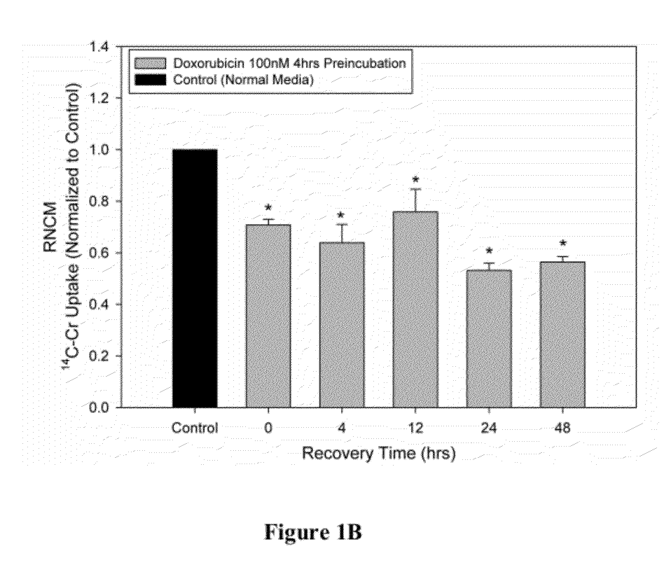

The effects of DOX on Cr transport capacity were quantified by measuring ¹⁴C—Cr uptake in primary RNCMs. Cr transport decreased significantly within 2-4 hrs of exposure to 100 nmol/L DOX (FIG. 1A). To determine if the effect of DOX on Cr transport remained after DOX was removed from the culture media, Cr transport was measured at increasing time intervals after an initial exposure of 4 hrs to 100 nmol/L DOX. Cr transport activity did not recover even after 48 hrs of growth in media devoid of DOX (FIG. 1B).

DOX Effects on Cr Transport in HL-1 Cells were Dose and Time Dependent:

HL-1 cells were used to extend our investigations of the effects of DOX on Cr transport. As mentioned previously, these cells are a well-established immortalized murine atrial cell line with very low native Cr transport that retain the essential hallmarks of primary cardiomyocytes and are a proven expression system to study protein structure, function, and modulation (Nash S. et al., Receptors Channels 2: 165-174, 1994, White S. M. et al., Am J Physiol Heart Circ Physiol 286: H823-829, 2004). Cr transport measured in HL-1 cells expressing the human CrT protein followed Michaelis-Menten kinetics with a Vₘₐₓ of 42.9±5.24 nmol/mg protein and Kₘ of 63.5±2.37 μmol/L. These values are equivalent to those reported for Cr transport in other mammalian expression systems (Darrabie M. D. et al., Am J Physiol Endocrinol Metab 300: E870-876, 2011, Nash S. et al., Receptors Channels 2: 165-174, 1994).

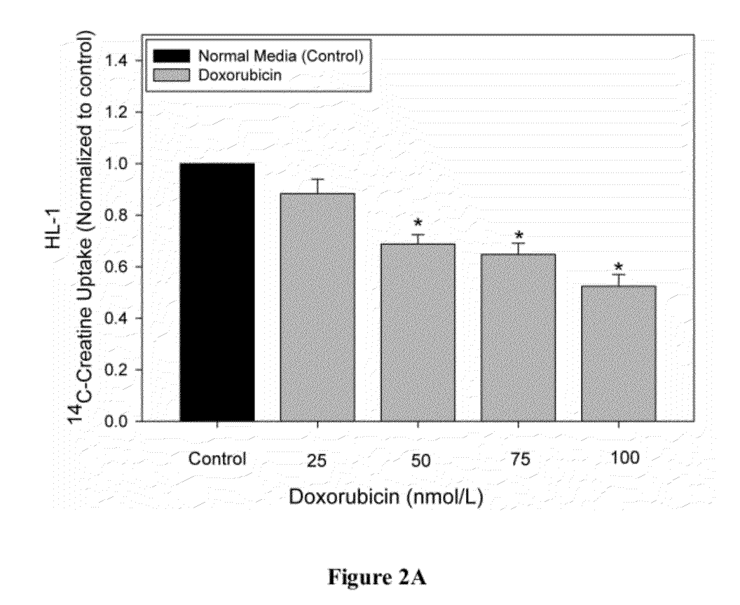

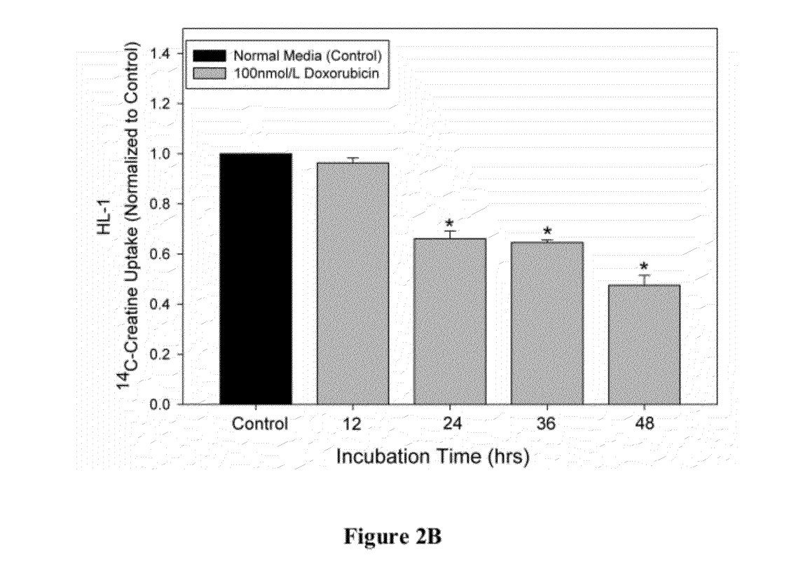

The dose dependence of DOX's effect on Cr transport was measured in HL-1 cells following incubation for 24 hrs in media containing 25, 50, 75 or 100 nmol/L DOX. The decrease in Cr transport induced by incubation with DOX was dose dependent. A significant decrease in Cr transport was observed in cells incubated for 24 hrs in media containing DOX concentrations as low as 50 nmol/L (FIG. 2A). The time dependence of DOX's effects on Cr transport in HL-1 cells expressing the CrT protein was examined by incubating cultures in 100 nmol/L DOX for 12, 24, 36, and 48 hrs (FIG. 2B). Cr transport decreased by 34.2% (p<0.05) after 24 hrs of exposure and continued to fall through 48 hrs of observation, when transport was then maximally decreased by 53.5% (p<0.05) relative to controls.

Changes in Cr Transport were not Due to Cell Death:

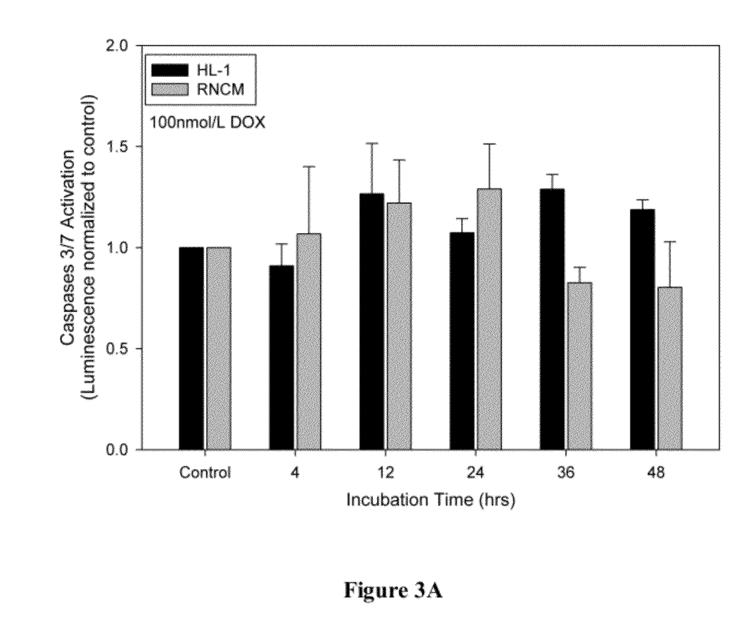

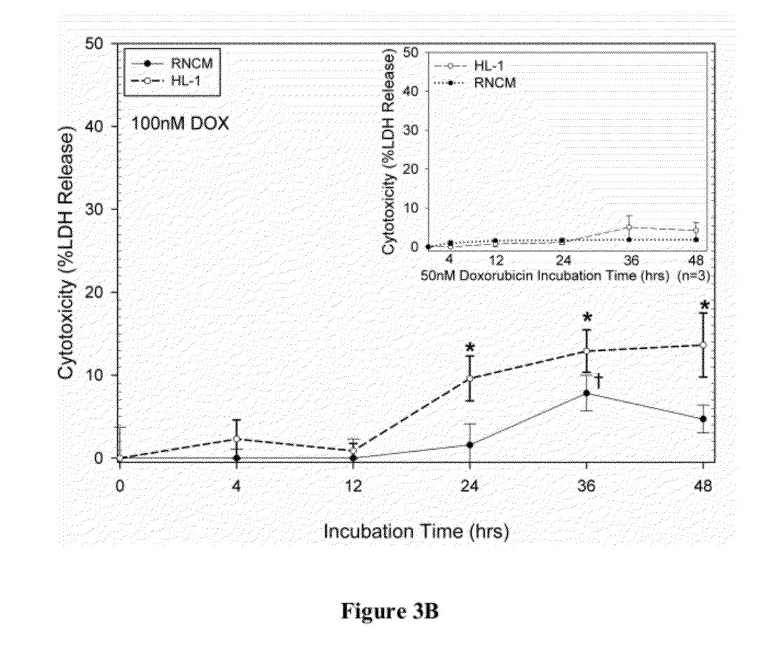

To determine the contribution of DOX induced cellular injury to the reduction in Cr transport observed in RNCM or HL-1 cultures, apoptosis and cytotoxicity were measured in cells incubated with 100 nmol/L DOX—double the concentration where significant decreases in Cr uptake were evident (FIG. 2A). Apoptosis was assessed by quantification of caspase 3/7 activation. No increase in apoptosis was detected in RNCM or HL-1 cultures incubated with 100 nmol/L DOX for up to 48 hrs (FIG. 3A).

Cytotoxicity was quantified using LDH release assays. 24 to 36 hrs elapsed before any changes in LDH release were detected in RNCM cells incubated in 100 nmol/L of DOX. A small increase in LDH release (7% compared with control, p<0.05) was detected in RNCM cells after 36 hrs of exposure to 100 nmol/L DOX (FIG. 3B), 32 hrs after Cr uptake had decreased by 37% (FIG. 1A).

In HL-1 cells, 24 hrs incubation with the highest dose of DOX studied (100 nmol/L) (FIG. 3B) increased LDH release by approximately 10% compared with controls. This dose of DOX decreased Cr uptake by 34% to 42% (FIGS. 2A and 2B), compared with the 31% reduction measured in these cells when they were exposed to 50 nmol/L of DOX (FIG. 2A), a dose not associated with increased cell death in HL-1 nor RNCM cultures (FIG. 3B, insert).

DOX Reduced the Vₘₐₓ and Kₘ for Cr Transport and the Amount of CrT Protein at the Cell Surface:

Kinetic analysis of Cr transport measured in HL-1 cells following 12 or 24 hrs of incubation with 100 nmol/L DOX demonstrated a significant reduction in Vₘₐₓ. A significant reduction in Kₘ was also observed after 24 hrs of incubation with DOX (see Table 1 below). To address the possibility that the reduction in Cr transport was due to DOX competing for transport with Cr, we determined Vₘₐₓ and Kₘ after ¹⁴C—Cr uptake assays in the presence of 2 μmol/L DOX or 2 μmol/L DOX and 100 μmol/L B-GPA, a structural analogue of Cr, that inhibits Cr transport with an IC₅₀ of 50 μmol/L (Dai W. et al., Arch Biochem Biophys 361: 75-84, 1999). Acute exposure to DOX did not significantly alter Cr transport Vₘₐₓ or Kₘ (see Table 2 below), demonstrating that DOX does not compete with Cr for transport by CrT.

TABLE 1
Effect of DOX on Creatine (Cr) transport Vₘₐₓ and Kₘ

| Condition | Relative Kₘ ± SEM | Relative Vₘₐₓ ± SEM |
|---|---|---|
| Control | 1.00 | 1.00 |
| 12 h DOX | 0.83 ± 0.09 | 0.75 ± 0.08* |
| 24 h DOX | 0.75 ± 0.08* | 0.63 ± 0.08* |

n = 3,
*p < 0.05, ANOVA, Fisher LSD Test

TABLE 2
DOX does not compete with Creatine (Cr) for transport

| Condition | Relative Vₘₐₓ ± SEM | Relative Kₘ ± SEM |
|---|---|---|
| Control | 1.00 | 1.00 |
| DOX | 1.09 ± 0.36 | 1.76 ± 0.69 |
| β-GPA | 0.43 ± 0.11 | 23.01 ± 3.04* |
| β-GPA + DOX | 0.47 ± 0.23 | 22.40 ± 5.95* |

n = 3,
*p < 0.05 ANOVA, Fisher LSD, compared to Control and DOX only

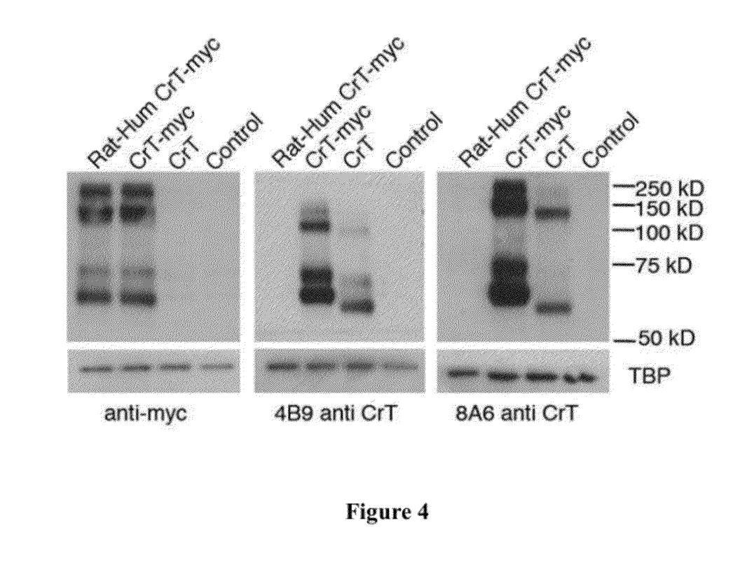

Changes in Vₘₐₓ can indicate changes in the cell surface population of membrane transporter protein (Qian Y. et al., J Neurosci 17: 45-57, 1997; Zapata A. et al., J Biol Chem 282: 35842-35854, 2007). Thus, the amount of CrT in the cell membrane was determined using cell-surface biotinylation followed by avidin affinity binding. This approach has successfully been used to identify the cell membrane dwelling population of CrT protein (Darrabie M D et al., Am J Physiol Endocrinol Metab 300: E870-876, 2011; Li H. et al., Am J Physiol Renal Physiol 299: F167-177, 2010). CrT protein was detected by Western blotting using human-specific rat monoclonal antibodies that were generated using genetic immunization technology as described herein above. Several distinct antibody clones detected the N-terminus of the human isoform of the CrT, with a 55 kD band corresponding to the monomer and cell-surface CrT protein. The heavier CrT bands likely represent adducts (multimers) of the transporter protein. No CrT specific bands were detected in cells transfected with a negative control, or in the cells that expressed a chimeric rat/human CrT protein where the N-terminus was identical to that of the rat isoform (FIG. 4).

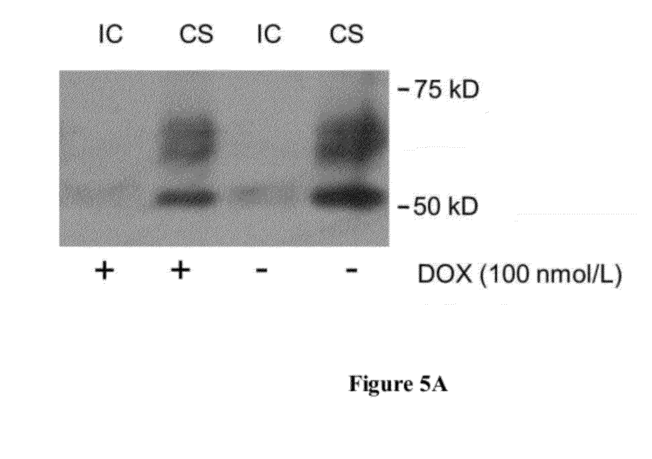

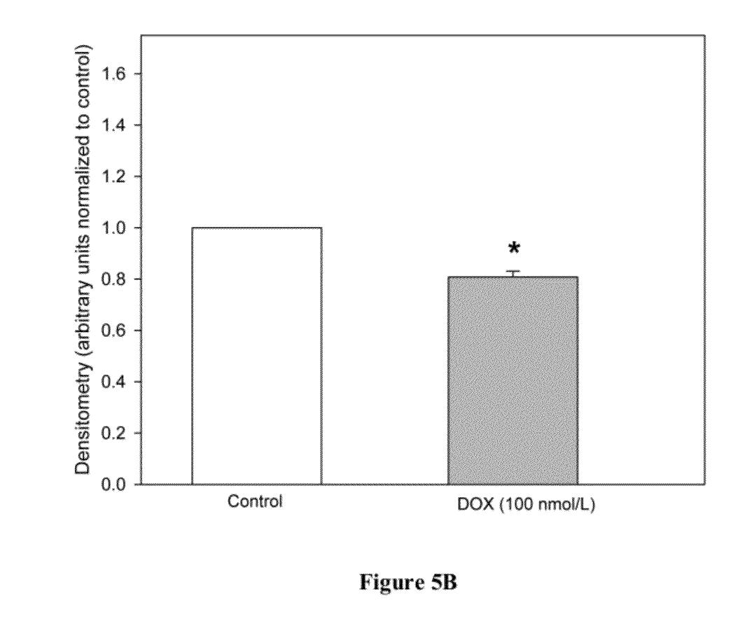

The amount of CrT at the cell surface was decreased in HL-1 cell cultures grown in media with 100 nmol/L DOX (FIGS. 5A and 5B). These changes in CrT protein mirrored the decrease in Cr transport capacity observed after exposure to DOX; there was a positive correlation (r=0.69, p<0.05, n=12) between Vₘₐₓ (Table 2) and cell-surface CrT protein abundance.

DOX Effects on Intracellular Cr and High-Energy Phosphate Levels:

The effects of DOX on intracellular content of Cr, PCr, ATP, ADP and AMP were quantified by LCMS/MS analysis performed on extracts prepared from HL-1 cultures incubated in the presence of 100 nmol/L DOX for 24 hrs and compared with data obtained from control cultures. The only significant difference measured was a decrease in AMP in cultures exposed to DOX (see Table 3 below).

TABLE 3
Quantification of Cr, PCr, ATP, ADP, and AMP

| | Cr | PCr | ATP | ADP | AMP |
|---|---|---|---|---|---|
| | (μmols/mg prot) | (μmols/mg prot) | (μmols/mg prot) | (μmols/mg prot) | (μmols/mg prot) |
| Control | 23.75 ± 2.47 | 6.43 ± 1.21 | 0.74 ± 0.20 | 0.60 ± 0.07 | 0.54 ± 0.05 |
| DOX | 23.01 ± 7.92 | 5.94 ± 0.12 | 0.60 ± 0.15 | 0.50 ± 0.07 | 0.26 ± 0.08* |

n = 3,
*significant compared to control, t-test, p ≦ 0.05

### Example 2

### Immunohistochemistry for Antibodies Specific for Creatine Transporter Protein

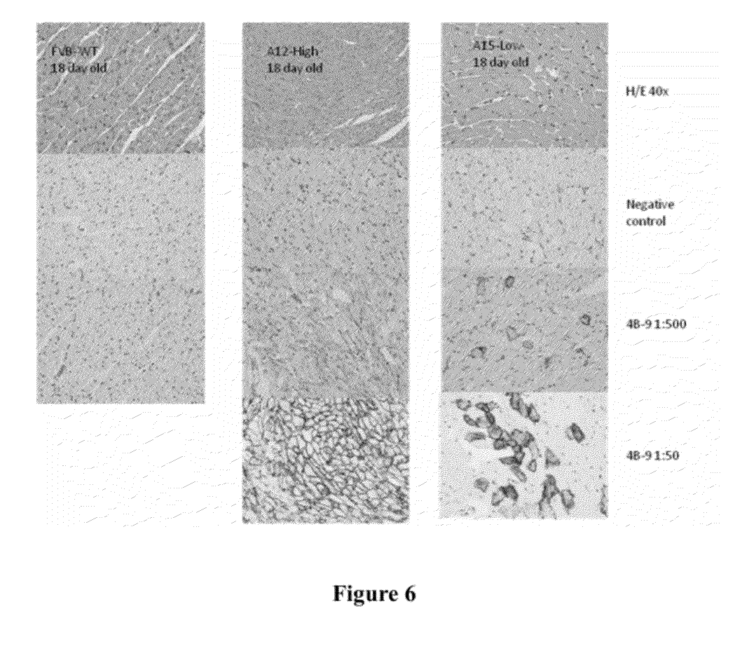

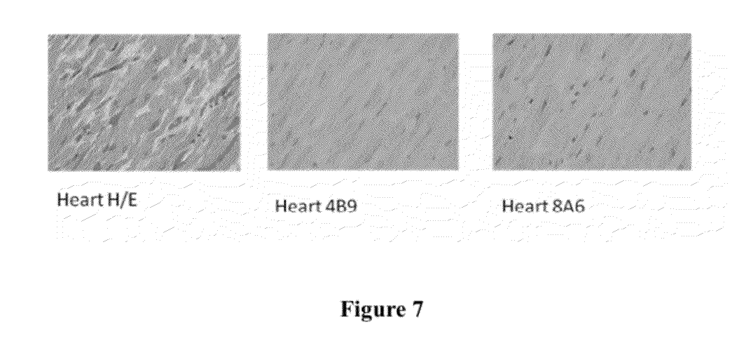

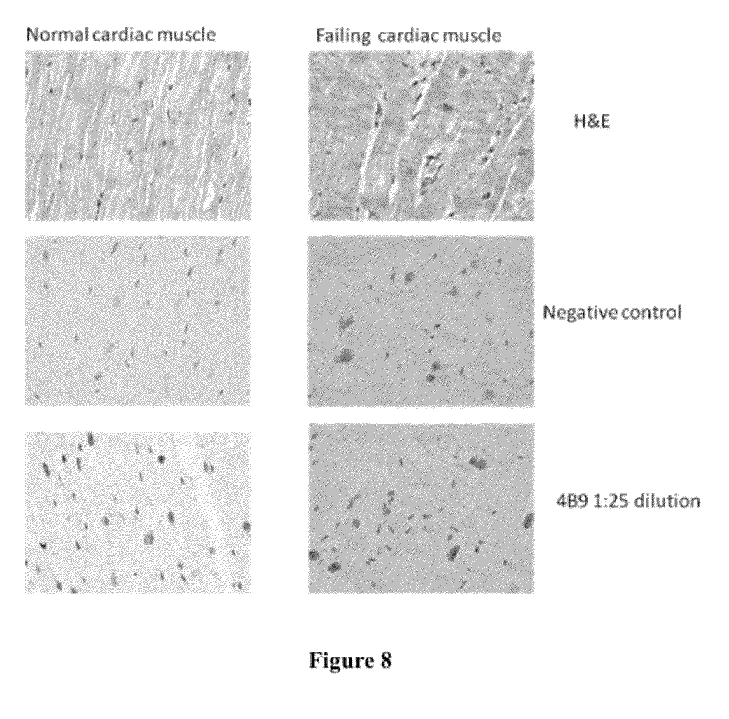

Formalin-fixed tissue samples of cardiac tissue obtained from (i) transgenic mice overexpressing the human creatine transporter protein (CrT) (FIG. 6); (ii) human subjects (FIG. 7); or (iii) human subjects having either normal cardiac tissue or failing cardiac tissue (tissues were obtained under an approved IRB protocol) (FIG. 8) were embedded in paraffin. Four micrometer sections were cut from the tissue blocks, placed on positive charged slides, allowed to dry, and then heated in a 65° C. oven for thirty minutes. After removal of paraffin with xylene and clearing with alcohol, the slides were placed in hydrogen peroxide and methanol to quench endogenous peroxidase activity. Sections were hydrated and washed in deionized water. It was determined during antibody optimization that pretreatment with proteinase K (DAKO concentrate diluted to 0.05 ml in 1.0 ml of 0.05M Tris, pH 7.5) was the proteolytic enzyme of choice. Comparative tissue pretreatment studies were performed using heat induced epitope retrieval. The tissue sections were digested for five minutes in the proteinase K solution then rinsed in deionized water and placed in Tris buffered saline (TBS) pH 7.5. Primary antibody rat monoclonal anti-4B9 or anti-8A6 at dilutions of 1:25 to 1:500 were applied and incubated for one hour at room temperature (the 4B9 and 8A6 antibodies were generated as described herein above at Example 1 in the section Creatine Transporter Protein (CrT) Antibody Generation). Following a rinse and wash with TBS, the bound primary antibody was linked with biotinylated goat anti-rat IgG (H&L specific, 5 μg/ml, VECTOR LABORATORIES, Burlingame, Calif.). The formed immune complex was further amplified and labeled with horseradish peroxidase conjugated avidin biotin complex (ABC ELITE, VECTOR LABS). Diaminobenzidine (DAB) was used to visualize the complex consisting of the bound anti CrT antibodies-biotin-avidin horseradish peroxidase The slides were washed with tap water, hematoxylin counter stain was applied, followed by dehydration with absolute alcohol, cleared with xylene and cover slipped with a permanent mounting media.

### Example 3

### Measurement of Creatine Transport in Erythrocytes

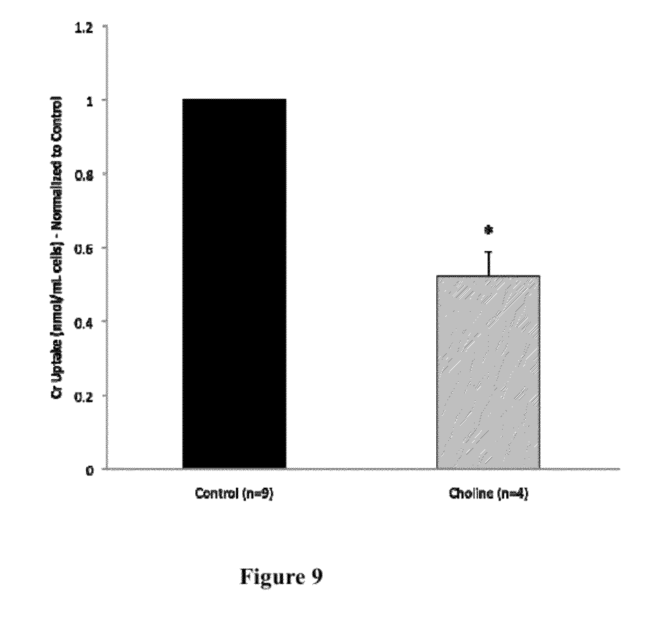

A sample of human whole blood was collected and creatine transport was quantified according to the following procedure. First, whole blood (10 ml) was centrifuged at 1500 g for 5 minutes and white nucleated elements were removed by aspiration of the buffy coat (Berthiaume J. M. & Wallace K. B., Cell Biol Toxicol 23: 15-25, 2007). Cells were resuspended in RPMI (INVITROGEN, Carlsbad, Calif.) media and plated in 24 well culture plates. To measure creatine transport, cells were centrifuged in the culture dishes at 1500 g, the supernatant was removed by aspiration, and the cells were resuspended in room temperature choline buffer (150 mmol/L choline chloride; 1 mmol/L CaCl₂; 5 mmol/L MgCl₂; 2 mmol/L KCl; and 5 mmol/L HEPES-Tris, pH 7.5). This procedure was repeated once more. After the last centrifugation, the cells were resuspended in sodium uptake buffer (150 mmol/L NaCl; 1 mmol/L CaCl₂; 5 mmol/L MgCl₂; 2 mmol/L KCl; and 5 mmol/L HEPES-Tris, pH 7.5) that was supplemented with 0.275 μCi/ml of ¹⁴C—Cr (55 mCi/mmol, AMERICAN RADIOLABELED CHEMICALS, St Louis, Mo.) and unlabeled Cr to a final concentration of 15 μmol/L for the dose-response and time course experiments, which is within the reported physiological range (Persky A. M., et al., J Clin Pharmacol 43: 29-37, 2003). Cells were then incubated for 10 min in a CO₂ incubator at 37° C. Uptake was terminated by removal of the radiolabeled solution by centrifugation as described above and three washes in ice cold choline buffer also as described above. Since creatine transport depends on the physiological sodium gradient, non-specific creatine uptake was measured in cells that were incubated in choline buffer supplemented with radiolabeled creatine. Data obtained following these experimental conditions represents non-specific creatine binding/absorption that does not involve the CrT. The cells were lysed with 500 mmol/L NaOH and heated to 80° C. for 30 min. 100 μl of cell lysate was subjected to scintillation counting using a BECKMAN COULTER LS 6500 scintillation counter. Cr transport was normalized to cell number, determined by manual counting using a hematocytometer. The results are shown in FIG. 9.

### Example 4

### Immunoflorescent Labeling of Human Erythrocytes with Creatine Tranporter Specific Antibody

Creatine transporter protein present on the surface of erythrocytes from human whole blood samples was detected using a monoclonal antibody specific for creatine transporter protein. First, the cells were collected and processed prior to antibody detection according to the following procedure. Whole blood was centrifuged at 1500 g for 5 minutes and white nucleated elements were removed by aspiration of the buffy coat. Isolated erythrocytes were smeared on glass slides and let dry at room temperature, then fixed with 100% methanol for 30 minutes. Erythrocytes were probed overnight with 1:100 4B9 or 8A6 (the antibodies were generated as described herein above at Example 1 in the section Creatine Transporter Protein (CrT) Antibody Generation). The slides were washed three times with PBS, before being probed for 1 hour with a 1:500 dilution of ALEXA FLUOR-GOAT anti-rat IgG secondary antibody, and washed again. Erythrocytes probed with only the secondary antibody were used as a negative control.

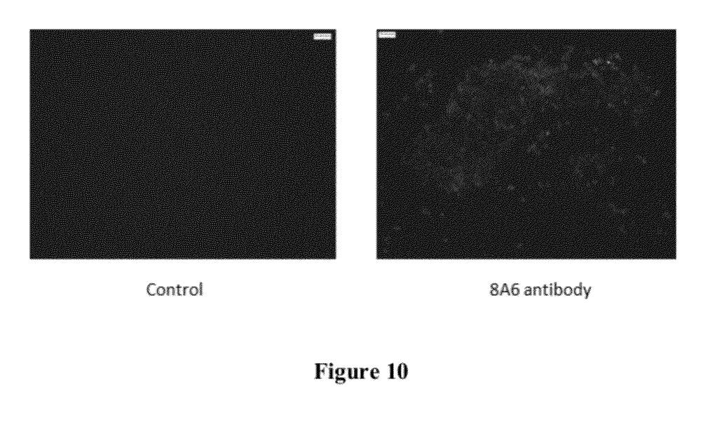

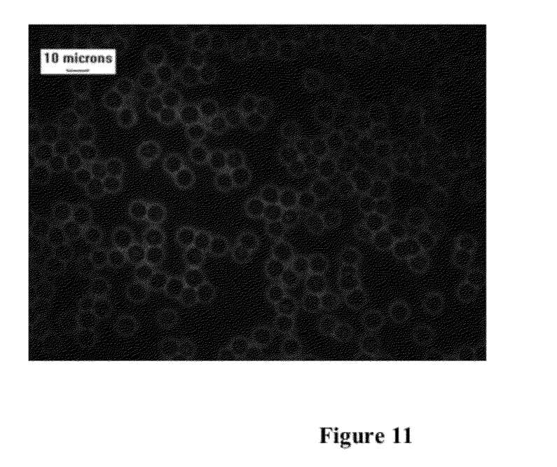

Slides were visualized and photographed using a fluorescence microscope, with the blue 470 nm or 490 nm filter (see FIG. 10). Erythrocytes shown in FIG. 10 were immunolabeled using 8A6 antibody, whereas those shown in FIG. 11 were labeled using 4B9 antibody. The image in FIG. 11 is magnified to depict in greater detail the fluorescent signal on the cell surface of the erythrocytes.

Variations and modifications of the herein described systems, apparatuses, methods and other applications will undoubtedly suggest themselves to those skilled in the art. Accordingly, the foregoing description should be taken as illustrative and not in a limiting sense.

Any patents or publications mentioned in this specification are indicative of the levels of those skilled in the art to which the invention pertains. These patents and publications are herein incorporated by reference to the same extent as if each individual publication was specifically and individually indicated to be incorporated by reference.

What is claimed:

1. A method for detecting cardiotoxicity in a subject undergoing treatment with an anthracycline compound comprising:
contacting a biological sample obtained from the subject with a monoclonal antibody that is specific for the creatine transporter protein; and
determining the level of binding of the antibody to the creatine transporter protein in the biological sample; wherein a lowered binding of antibody as compared to a control is indicative of cardiotoxicity.

2. The method of claim 1, wherein the antibody recognizes the isoform of creatine transporter protein that is recognized by the antibody produced by hybridoma cell line 4B9 or 8A6.

3. The method of claim 1, wherein the antibody is specific for an epitope in the first 60 amino acids of the human creatine transporter protein (SEQ ID NO: 1).

4. The method of claim 1, wherein the antibody comprises antibody produced by hybridoma cell line 4B9 or hybridoma cell line 8A6.

5. The method of claim 1, wherein the level of antibody binding is determined by one or more of flow cytometry, immunohistochemistry, ELISA, or Western blotting.

6. The method of claim 1, wherein the control is a control sample obtained from a biological sample from an individual not undergoing treatment with an anthracycline compound, the method comprising contacting the control sample with the monoclonal antibody that is specific for the creatine transporter protein; and determining the level of binding of the antibody to the creatine transporter protein in the control sample.

7. The method of claim 1, wherein the control is the biological sample obtained from the individual prior to beginning treatment with the anthracycline compound.

8. The method of claim 1 wherein the biological sample comprises blood or body tissue.

9. A method for detecting cardiotoxicity in a subject susceptible to a heart-related condition comprising:
contacting a biological sample obtained from the subject with a monoclonal antibody that is specific for the creatine transporter protein; and
determining the level of binding of the antibody to the creatine transporter protein in the biological sample; wherein a lowered binding of antibody as compared to a control is indicative of cardiotoxicity.

10. The method of claim 9, wherein the antibody recognizes the isoform of creatine transporter protein that is recognized by the antibody produced by hybridoma cell line 4B9 or 8A6.

11. The method of claim 9, wherein the antibody is specific for an epitope in the first 60 amino acids of the human creatine transporter protein (SEQ ID NO: 1).

12. The method of claim 9, wherein the antibody comprises antibody produced by hybridoma cell line 4B9 or hybridoma cell line 8A6.

13. The method of claim 9, wherein the level of antibody binding is determined by one or more of flow cytometry, immunohistochemistry, ELISA, or Western blotting.

14. The method of claim 9, wherein the control is a sample obtained from a biological sample from an individual not susceptible to a heart-related condition, the method comprising contacting the control sample with the monoclonal antibody that is specific for the creatine transporter protein; and determining the level of binding of the antibody to the creatine transporter protein in the control sample.

15. The method according to claim 9 wherein the biological sample comprises blood or body tissue.

16. A kit for determining the likelihood of the presence of cardiotoxicity in a subject, the kit comprising (i) an antibody that specifically binds to the creatine transporter protein and (ii) instructions for use in a method of determining the likelihood of the presence of cardiotoxicity in a subject, the method comprising: contacting a biological sample obtained from the subject with a monoclonal antibody that is specific for the creatine transporter protein; and determining the level of binding of the antibody to the creatine transporter protein in the biological sample, wherein a lowered binding of antibody as compared to a control is indicative of cardiotoxicity.

17. The kit of claim 16, wherein the antibody recognizes the isoform of creatine transporter protein that is recognized by the antibody produced by hybridoma cell line 4B9 or 8A6.

18. The kit of claim 16, wherein the antibody is specific for an epitope in the first 60 amino acids of the human creatine transporter protein (SEQ ID NO: 1).

19. The kit of claim 16, wherein the antibody comprises antibody produced by hybridoma cell line 4B9 or hybridoma cell line 8A6.

20. The kit of claim 16, comprising one or more of a buffer, a secondary antibody, or a detection reagent.

21. A method for detecting the onset of cardiotoxicity in a subject undergoing treatment with an anthracycline compound comprising:
obtaining a blood sample from a subject prior to treatment of the subject with an anthracycline compound and measuring creatine transporter protein activity in the blood sample erthyrocytes;
obtaining a blood sample from the subject after receiving at least one treatment with the anthracycline compound and measuring creatine transporter protein activity in the blood sample erthyrocytes;
comparing the level of creatine transporter protein activity in the erthyrocytes, wherein a reduced creatine transporter protein activity in the erthyrocytes after treatment with the anthracycline compound is indicative of the onset of cardiotoxicity; and
ceasing treatment with the anthracycline compound when the reduced creatine transporter protein activity is observed.
